(19) United States
(12) Patent Application Publication
(10) Pub. No.: US 2023/0203535 A1
(43) Pub. Date: Jun. 29, 2023

(54) Recombinant P5 Promoter for Use in Reducing DNA Contamination of AAV Preparations

(71) Applicant: ST. JUDE CHILDREN'S RESEARCH HOSPITAL, INC., Memphis, TN (US)

(72) Inventors: Mark BRIMBLE, Memphis, TN (US); Andrew DAVIDOFF, Memphis, TN (US); Pei-Hsin CHENG, Memphis, TN (US); Christopher MORTON, Memphis, TN (US)

(21) Appl. No.: 17/927,038

(22) PCT Filed: May 24, 2021
(86) PCT No.: PCT/US2021/033826

§ 371 Date: Nov. 22, 2022

Related U.S. Application Data
(60) Provisional application No. 63/029,839, filed on May 26, 2020.

(51) Int. Cl. C12N 15/86

(52) U.S. Cl. CPC C12N 15/86; C12N 2710/20022; C12N 2750/14143

(57) ABSTRACT

A recombinant P5 promoter having an insertion of an exogenous spacer between the REP binding site and a transcription start site-localized Ying-Yang 1 (YY1) binding site is described, wherein said recombinant P5 promoter provides for a reduction in DNA contamination upstream of the P5 promoter without a concomitant reduction in viral titer.

### INTRODUCTION

This application claims priority to U.S. Provisional Application No. 63/029,839, filed May 26, 2020, the content of which is incorporated herein by reference in its entirety.

### BACKGROUND

Adeno-Associated virus (AAV) is a small Parvovirus of the Dependovirus family and is one of the most commonly used gene therapy vectors. Clinical successes in the treatment of diseases such as Haemophilia, Leber’s Congenital Amaurosis, and Spinal Muscular Atrophy have increased interest in AAV-mediated gene therapy as a viable treatment strategy. However, there are still issues within the field that need to be monitored and addressed as clinical implementation of AAV-based therapies progress.

Post-treatment immunogenic responses have been observed in several trials and in certain research models dose-dependent toxicity has been reported. Furthermore, while AAV predominantly exists in an episomal state post-infection, integration into the host genome is still observed. Another issue of importance to the field is the purity of AAV vectors. Heterogeneity of DNA in recombinant AAV has been seen when attempting to package short hairpins and oversized constructs. Moreover, the incorporation of contaminating DNA from production plasmids has been observed in AAV in multiple production contexts. Previous efforts to remove these contaminants have focused on design interventions such as increasing the size of the expression cassette backbone plasmid and the implementation of DNA minicircles to the production process. However, there has been comparatively little investigation into the cause and effects of these DNA impurities.

While replacing the P5 promoter with the HIV-LTR promoter or a combination of the CMV promoter and metallothionein promoter has been suggested (see US 5,658,776 and Allen, et al. ((2000) Mol. Ther. 1(1):88-95), there was no indication of a change in the level of DNA contamination. Likewise, while the insertion of a spacer located between the 3′ end of the AAV p5 promoter and the 5′ end of the REP gene start codon has been shown to increase REP protein expression (see WO 2019/217483), there was no indication of a change in the level of DNA contamination.

### SUMMARY OF THE INVENTION

This invention is a recombinant P5 promoter composed of a REP binding site and a transcription start site-localized Ying-Yang 1 (YY1) binding site, wherein said P5 promoter includes an exogenous spacer sequence (e.g., 5-100 nucleotides in length) inserted between the REP binding site and YY1 binding site. An adeno-associated virus (AAV) vector, e.g., AAV1, AAV2, AAV3, AAV4, AAV6, AAV7, AAV8, or AAV9, harboring the recombinant P5 promoter is also provided as a host cell containing the same. A method of reducing DNA contamination upstream of a P5 promoter using the recombinant P5 promoter is further provided.

This invention is also a recombinant AAV harboring an HPV P97 promoter containing a REP binding site and a transcription start site-localized YY1 binding site.

### BRIEF DESCRIPTION OF THE DRAWINGS

FIG. 1 depicts the 3′ portion of the P5 promoter sequence (reference nucleotides 255-292 of AAV8) which includes the TATA box, REP binding site and Ying-Yang 1 (YY1) binding site. Imperfect GAGC repeats of the REP binding site are indicated with arrow. Bent arrow indicates transcription start site.

FIG. 2 shows that contaminant sequences closer to the inverted terminal repeats (ITRs) are more abundant than more remote sequences.

FIG. 3 shows contaminant sequences closer to the P5 REP binding site are present at greater levels than more distal sequences.

FIG. 4 shows titers of FVIII when the P5 promoter of a AAV2_8 REPCAP construct is replaced with an HPV16-P97 promoter, a HPV18-P105 promoter or the BPV upstream regulatory region (URR). qPCR titer analysis showed that all P5 replacement vectors tested, except for the HPV18-P105 construct, could produce AAV.

FIG. 5 shows the wild-type P5 structure (SEQ ID NO:7) and structure of P5 vectors containing either a mutation in the TRS mimic of the YY1 box (P5ΔTRS; SEQ ID NO:8) or the introduction of a spacer sequence between the YY1 box (SEQ ID NO: 9) and the P5 REP binding site (SEQ ID NO: 10) (P5-HS system).

FIG. 6 shows that P5-HS vectors with 5 bp (P5-HS5) and 100 bp (P5-HS100) spacer insertions yielded viral titers equivalent to that of the standard P5 promoter configuration (**P>0.9999).

FIG. 7 shows a comparison (One-way ANOVA with Sidak’s multiple comparisons test) of AAV1 and AAV2 titer produced with P5 or P5-HS5 promoters as determined by qPCR (n=3, P>0.7).

FIG. 8 shows a comparison of upstream contamination of AAV1 and AAV2 when using P5 or P5-HS5 promoters (n=3, **P<0.025).

### DETAILED DESCRIPTION OF THE INVENTION

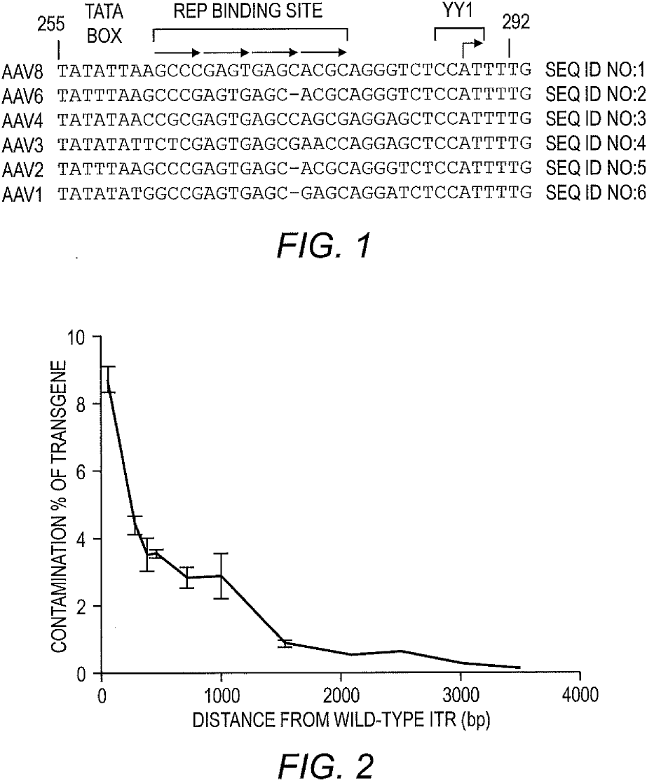

The complexity of the AAV life cycle is reflected in the regulation of the viral promoters by proteins produced by the helper virus and the host cell, as well as by AAV. Consequently, several known and putative cis elements for both viral and host trans activators and repressors have been localized in the AAV P5 promoter. These include cis elements such as the binding sites for CREB/ATF and major late transcription factor (MLTF; USF) in the region upstream of the P5 TATA box. The P5 promoter also contains two binding sites for the host cell transcription factor Ying-Yang 1 (YY1). One is located upstream from the TATA box, where YY1 binding results in repression of P5 expression in the absence of helper virus. In the presence of E1a protein produced by the coinfecting adenovirus, this site mediates transcription activation of the P5 promoter by a direct E1a-YY1 interaction. Binding of YY1 to the second binding site located at the transcription start site enhances and directs the accurate positioning of P5 transcript initiation. An additional element of the P5 promoter is a REP binding site, composed of four imperfect GAGC repeats, which is located just upstream of the +1 YY1-binding sequence (FIG. 1) and controls REP expression.

The present disclosure is predicated on the discovery that the insertion of a spacer between the P5 REP binding sequence and the YY1 box at the transcription start site of the P5 promoter eliminates active contaminant incorporation from a REP CAP plasmid, while maintaining recombinant AAV titers. Accordingly, this invention provides a recombinant P5 promoter composed of a REP binding site and a transcription start site-localized YY1 binding site, wherein the P5 promoter includes an exogenous spacer sequence inserted between the REP binding site and YY1 binding site. Excluding the exogenous spacer, the P5 promoter of the present disclosure includes a sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to wild-type P5 promoter. In some examples, the P5 promoter of the invention, into which the exogenous spacer is inserted, has a sequence of nucleotides corresponding to at least about 40, 50, 60, 70, 80, 90, 100, 110, 120, 130, 140, 150, or 160 nucleotides adjacent (or upstream of) the transcription start site of the large REP proteins (i.e., REP78 and REP68) in an AAV genome, or a sequence having at least 80%, 81%, 82%, 83%, 84%, 85%, 86%, 87%, 88%, 89%, 90%, 91%, 92%, 93%, 95%, 96%, 97%, 98% or more sequence identity thereto, wherein the promoter drives transcription of the large REP proteins, i.e., REP78 and REP68. In some examples, the promoter has a sequence derived from the sequence of the region adjacent to the transcription start site of REP78 and REP68 in AAV serotype 1 (AAV1), AAV serotype 2 (AAV2), AAV serotype 3 (AAV3), AAV serotype 4 (AAV4), AAV serotype 6 (AAV6), AAV serotype 7 (AAV7), AAV serotype 8 (AAV8) or AAV serotype 9 (AAV9). Amongst the P5 promoters provided herein are those that comprise or consist of a sequence corresponding to the approximately 160 nucleotides adjacent to the transcription start site of REP78 and REP68 in an AAV genome, such as an AAV1, AAV2, AAV3, AAV4, AAV6, AAV7, AAV8 or AAV9 genome.

The P5 promoter of the present invention can be provided as an isolated polynucleotide or part of an isolated polynucleotide. Accordingly, the present disclosure also provides isolated polynucleotides comprising a promoter described herein. As would be appreciated by those skilled in the art, the isolated polynucleotides may further contain one or more additional elements or sequences, such as any described herein or known in the art.

“REP binding sequence,” “REP binding site” or “REP binding element” are used interchangeably herein to refer to an element of the P5 promoter composed of two to four imperfect GAGC repeats that are located just downstream, i.e., 3′, of the TATA box and upstream of the transcription start site of the P5 promoter. See FIG. 1. Rep-mediated repression is thought to be the result of Rep binding to the REP binding element.

The “Ying-Yang 1 binding site,” “YY1 binding site” or “YY1 box” refers to a YY1 recognition sequence, which when bound by YY1, supports initiation from the transcription start site. The +1 YY1 binding of the P5 promoter is located at the transcription start site and has the consensus sequence CCAT.

In accordance with this invention, the P5 promoter includes an exogenous spacer sequence between the REP binding site and YY1 binding site. As used herein, the term “exogenous spacer sequence” refers to a sequence that is not native to the P5 promoter. The exogenous spacer sequence may be inserted anywhere between the REP binding site and YY1 binding site as described herein. In some embodiments, the exogenous spacer sequence is inserted into or replaces one or more of the seven nucleotides located between the REP binding site and YY1 binding site. By way of illustration, the exogenous spacer sequence may be inserted into or replace one or more nucleotides of the sequence AGGGTCT of the AAV8 P5 promoter.

In some embodiments, the total length of the exogenous spacer is 5, 10, 15, 20, 25, 30, 35, 40, 45, 50, 55, 60, 65, 70, 75, 80, 85, 90, 95, 100, 105, 110, 115, 120 or more nucleotides, or a length within any range delimited by any pair of the foregoing values, such as between 5 and 120, between 10 and 100 or between 5 and 100, for example. Ideally, the exogenous spacer is 5 to 100 nucleotides in length. As would be understood by those skilled in the art, the sequence of the spacer is not critical. The sequence of the spacer may be a random, artificial sequence, a sequence derived from AAV or a sequence derived from another source.

The present disclosure also provides vectors comprising a recombinant P5 promoter described herein. The recombinant P5 promoter of the present disclosure can be included in any suitable vector to regulate or drive transcription of the large REP proteins, i.e., REP78 and REP68. In some embodiments, the vector is a viral vector. Ideally, the vector is an AAV vector.

In certain embodiments, the AAV vector is selected from the group of AAV1, AAV2, AAV3, AAV4, AAV6, AAV7, AAV8, or AAV9. The vector can be an episomal vector (i.e., a vector that does not integrate into the genome of a host cell) or can be a vector that integrate into the host cell genome. It is contemplated that any vector system using an AAV REP and AAV P5 promoter can include the P5 promoter of this invention. For example, the P5 promoter and AAV2 REP gene feedback loop is used in recombinant bocavirus production (Yan, et al. (2013) Mol. Ther. 21:2181-2194).

In some embodiments, the instant P5 promoter and AAV rep and cap genes are present in a plasmid. It is contemplated that the cap genes and/or rep gene are from any AAV serotype (including, but not limited to, AAV1, AAV2, AAV4, AAV5, AAV6, AAV7, AAV8, AAV9, and any variants thereof).

Vectors suitable for use in mammalian cells are widely described and well-known in the art. Those skilled in the art would appreciate that vectors of the present invention that comprise a promoter described herein will also contain additional sequences and elements useful for the replication of the vector in prokaryotic and/or eukaryotic cells, selection of the vector and the expression of the sequences in a variety of host cells. For example, the vectors of the present disclosure can include a prokaryotic replicon (that is, a sequence having the ability to direct autonomous replication and maintenance of the vector extrachromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof.

Also provided are recombinant virions, including recombinant AAV virions, produced using the viral vectors described herein. Most typically, the recombinant virions include a heterologous coding sequence operably linked to a promoter. As will be understood by those skilled in the art, in most instances, not all of the nucleotides of the viral vector will be packaged into the recombinant virus. For example, in the case of AAV vector packaging, only the ITRs and the nucleotides flanked by the ITRs, including a promoter as well as any other sequences downstream of the promoter and upstream of the 3′ ITR, such as a heterologous coding sequence, will be packaged into the recombinant AAV. The recombinant virions can be used to deliver the heterologous coding sequence to a host cell for expression in that cell.

Methods for packaging viral vectors to produce recombinant virions are well known in the art, and any such method can be used to produce recombinant virions. By way of illustration, methods for producing a recombinant AAV include introducing into a packaging cell line an AAV vector encoding a heterologous coding sequence, helper functions for generating a productive AAV infection, and AAV cap and rep genes, the latter of which includes a P5 promoter of the invention; and recovering a recombinant AAV from the supernatant of the packaging cell line. Various types of cells can be used as the packaging cell line. For example, packaging cell lines that can be used include, but are not limited to, HEK 293 cells, HeLa cells, and Vero cells, for example, as disclosed in US 2011/0201088.

The helper functions may be provided by one or more helper plasmids or helper viruses harboring adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US 2011/0201088, and helper vectors pHELP (Applied Viromics). A skilled artisan will appreciate that any helper virus or helper plasmid of AAV that can provide adequate helper function to AAV can be used herein.

In some instances, recombinant AAV is produced by using a cell line that stably expresses some of the necessary components for AAV virion production. For example, a plasmid (or multiple plasmids) including the instant P5 promoter, AAV rep and cap genes, and a selectable marker, such as a neomycin resistance gene, can be integrated into the genome of a cell (the packaging cells). The packaging cell line can then be co-infected with a helper virus (e.g., adenovirus providing the helper functions) and an AAV vector for expressing a heterologous gene of interest. The advantages of this method are that the cells are selectable and are suitable for large-scale production of the recombinant AAV. As another non-limiting example, adenovirus or baculovirus rather than plasmids can be used to introduce rep and cap genes into packaging cells. As yet another non-limiting example, both the AAV vector for expressing a heterologous gene of interest and the rep-cap genes (including the instant P5 promoter) can be stably integrated into the DNA of producer cells, and the helper functions can be provided by a wild-type adenovirus to produce the recombinant AAV.

As will be appreciated by a skilled artisan, any method suitable for purifying AAV can be used in the embodiments described herein to purify the recombinant AAV, and such methods are well known in the art. For example, the recombinant AAV can be isolated and purified from packaging cells and/or the supernatant of the packaging cells. In some embodiments, the AAV is purified by separation method using a CsCl gradient. In other embodiments, AAV is purified as described in US 2002/0136710 using a solid support that includes a matrix to which an artificial receptor or receptor-like molecule that mediates AAV attachment is immobilized.

Also provided herein are host cells comprising a vector or recombinant virion of the present disclosure. In some instances, the host cells are used to amplify, replicate, package and/or purify a vector or recombinant virion. In other examples, the host cells are used to express a heterologous coding sequence under the control of a suitable promoter. In some embodiments, the methods may be in vitro, ex vivo or in vivo.

Exemplary host cells include prokaryotic and eukaryotic cells. In some instances, the host cell is a mammalian host cell. In instances where the cells are used to package a viral vector described herein, the cells may also be transfected with one or more plasmids or infected with one or more viruses that provide the necessary helper and accessory molecules for packaging. In further example, the host cells may stably express, such as from the genome, one or more helper and accessory molecules. It is well within the skill of a skilled artisan to select an appropriate host cell for the amplification, replication, packaging and/or purification of a vector or recombinant virion of the present invention. Exemplary mammalian host cells include, but are not limited to, HEK-293 cells, HeLa cells, Vero cells, HUH7 cells, and HepG2 cells.

Also provided is a method of reducing DNA contamination upstream of a P5 promoter by inserting, between the REP binding site and YY1 binding site of the P5 promoter, an exogenous spacer sequence. As demonstrated herein, insertion of a 5 nucleotide to 100 nucleotide exogenous spacer between the REP and YY1 binding sites, results in the production of higher quality clinical AAV products while maintaining equivalent titers compared to conventional production systems. An advantage of this invention is that AAV can be produced in a cell transfection system without the incorporation of contaminant DNA upstream of the P5 promoter. Levels of contaminant DNA are a release criterion for AAV gene therapy trials, and reduction of contaminant DNA levels would improve both the purity of the vector, and the likelihood of a vector being cleared for trial.

In addition to the above-described P5 promoter, this invention also provides a recombinant AAV, wherein the P5 promoter driving the large REP proteins (REP78 and REP68) has been replaced with an HPV P97 promoter comprising a REP binding site and a transcription start site-localized YY1 binding site. As with the P5 promoter, a recombinant AAV harboring a P97 promoter upstream of the REP78 and REP68 coding sequences can be used in methods for producing AAV with reduced contaminant DNA.

The following non-limiting examples are provided to further illustrate the present invention.

### Example 1: Materials and Methods

Vectors and Sequences. P5 promoter modified vector fragments were synthesized by Genewiz and cloned into AAV2_8 REPCAP or AAV2_2 REPCAP backbones via NotI + SalI sticky end cloning. Green fluorescent protein (GFP) cassettes were cloned into a modified AAV2_8 REPCAP backbone by SpeI + XbaI sticky end cloning to yield forward and reverse direction constructs. All cloned constructs were sequence verified.

AAV Production, Purification and Titer. AAV8 FIX and FVIII viruses were produced by two plasmid transfections in CELLSTACK® culture chambers. Prior to transfection, adherent Human embryonic kidney (293T) cells (ATCC CRL-3216) were cultured in Dulbecco’s Modified Eagle Medium (DMEM, Lonza BioWhittaker) supplemented with 10% Fetal Bovine Serum (Fisher Scientific) and 2 mM L-Glutamine (Corning) at 37° C. and 10% CO₂. Transfection plasmids included: (a) a REP-CAP plasmid with AAV2_8 to provide the replication and capsid genes; (b) scLP1hFIXco+helpV3 to provide a self-complementary FIX expression cassette and adenoviral helper genes; and (c) ssHLPhFVIIIv3 to provide a single-stranded FVIII expression cassette with adenoviral helper genes. Transfection plasmids were resuspended in DMEM, and passed through a 0.2 µM filter into DMEM containing PEIPRO® transfection reagent (Polyplus). The DNA-PEI mixture was mixed, incubated for 15 minutes, and combined with 800 ml of media from the CELLSTACK® culture chamber. The resulting transfection mixture was then poured back into CELLSTACK® culture chamber and incubated at 37° C. and 10% CO₂. Supernatants were harvested 7 days post-transfection and concentrated to 15-30 ml using a multi-manifold PELLICON® XL50 cassette (Millipore-Sigma). Concentrated supernatants were treated with BENZONASE® nuclease (25 units/ml, Millipore-Sigma) in presence of 1 mM MgCl₂ at 37° C. for 1 hour. Treated supernatants were passed through a 0.2 µM filter, diluted in phosphate-buffered saline (PBS) and run on through a POROS™ CaptureSelect™ AAVX resin (Thermo Fisher Scientific) . Flow rate on the column was set to 2 ml/minute. The column was equilibrated with five column volumes of PBS pH 7.4 and AAV diluted supernatants were applied to the column. The column was then washed with 15 column volumes of PBS pH 7.4 and eluted with five column volumes of 0.1 M glycine-HCl, pH 2.7 followed by five column volumes of PBS pH 7.4. Elution fractions of 4 ml were collected. Fractions were titered by qPCR and peak fractions were combined. Eluted AAV was concentrated with a 100 KDa AMICON® filter (Millipore-Sigma) and 0.25% rHA was added to the formulation. Virus was stored at 4° C. short term and at -80° C. long term.

Small Scale AAV Production and Sample Preparation for Contaminant Analysis. Two µL of supernatant from a small scale AAV preparation were pretreated with 500 U DNase I in 1X NEBuffer 3 in a reaction volume of 100 µL at 37° C. for 1 hour. Reactions were neutralized with 2 µL 0.5 M EDTA pH 8 and incubated at 98° C. for 10 minutes. The solution was cooled and capsid proteins were denatured via addition of 2 µL 10% SDS and 2 µL of 20 mg/ml proteinase K (Ambion) for 1 hour at 55° C. Proteinase was inactivated at 98° C. for 1 hour and 94 µL of 0.01% Pluronic® F-68 non-ionic surfactant (Gibco) was added. Samples were then analyzed for production efficiency by quantitative PCR.

qPCR Titration of AAV Virus and Contaminant Amplicons. Expression cassette titer and contaminant amplicon titers were performed by qPCR using 1e-4 and 1e-5 serially diluted virus in ddH₂O + 0.005% PLURONIC® F-68 non-ionic surfactant (Gibco) against linearized, serially diluted plasmid standard using SYBR green technology (Applied Biosystems). Plate-to-plate variation in the contaminant titer experiments was accounted for via measurement and normalization of the expression cassette titer calculated from each plate to a predetermined value.

Deep Sequencing Analysis of AAV Virus. AAV genomes (1e11 viral genomes (vg)) were diluted in PBS to a volume of 200 µL and capsids were denatured at 95° C. for 15 minutes. Samples were cooled on ice and 117 uL of 100% EtOH was added to precipitate DNA. Virus was then passed through a QIAamp MinElute Virus spin kit (QIAGEN) and eluted in 25 µL ddH₂0. Purified DNA fragments were then subjected to next-generation sequencing (NGS) to obtain 15 million paired-end reads of 100 bp in length. Sequencing reads were then mapped back to the AAV production plasmids. For analysis of post-infection hepatocytes, 100 million paired-end reads were collected, reads that corresponded to the mouse genome were discarded and the remaining reads were mapped back to the AAV producer plasmids.

In Vivo Studies. For in vivo contamination studies, male C57BL/6 mice were injected via tail vein with 8.4e11vg AAV8. Upon termination of the experiment, livers were harvested and snap frozen in liquid nitrogen and stored at -80° C. Genomic hepatic DNA and RNA were isolated using commercial purification kits (QIAGEN). Genomic DNA was treated with RNAse A.

For in vivo P5-GFP flanker studies, mice were injected via tail vein with 4.11e11vg AAV8. One week after injection livers were harvested and formalin-fixed. Hematoxylin-eosin (H&E) slides were made and stained for GFP by immunohistochemistry. Slides were imaged on an EVOS® FL Auto (Life Technologies) microscope, on brightfield 20X magnification.

For therapeutic protein expression studies, C57BL/6 mice were injected with 2e10vg of AAV. Circulating clotting factor levels of FVIII or FIX were assessed by ELISA (Diagnostica Stago) on blood extract. ELISA analysis was performed on BioTek plate reader and all graphs were plotted in GraphPad Prism 7.0.

Cell Line Samples. Cell line samples were media aspirated, washed in 1X PBS, treated with trypsin and incubated for 5 minutes at 37° C. and 10% CO₂. Cell solutions were neutralized with D10 media and centrifuged at 400 g for 5 minutes. Cell samples were resuspended to a concentration of between 50,000 and 100,000 cells in 140 µL PBS. Samples were transferred into a cytospin sample chamber and centrifuged at 400 g onto a glass slide. Slides were incubated in 1% paraformaldehyde (PFA) in PBS with 0.05% Nonidet P-40 (MP Biomedicals) for 5 minutes, then in 1% PFA in PBS for 5 minutes, and then in 70% EtOH for 5 minutes before being transferred to storage in 70% EtOH at -20° C.

Fresh Liver Samples. Liver samples were cut with a razor blade to expose a fresh edge and touched onto slides (Thermo-Scientific). Slides were incubated in 1% PFA in PBS with 0.05% Nonidet P-40 (MP Biomedicals) for 5 minutes, then in 1% PFA in PBS for 5 minutes, and then in 70% EtOH for 5 minutes before being transferred to storage in 70% EtOH at -20° C.

Fluorescence in situ Hybridization (FISH). Slides were sequentially treated for 2 minutes each in 70%, 80%, and 100% EtOH solutions. Slides were then dried and probed for either expression cassette RNA or contaminant RNA. Denatured probes were prepared by nick translation and resuspended in buffer composed of 50% formamide, 2X saline-sodium citrate, and 10% dextran sulfate (Namekawa, et al. (2010) Mol. Cell. Biol. 30:3187-3205). Probe was applied to slides and hybridized at 37° C. overnight. Slides were then washed in 50% formamide and 2X saline-sodium citrate at 37° C. for 5 minutes. Slides were mounted in VECTASHIELD® mounting medium. RNA FISH images were collected and analyzed by standard methods (Paulk, et al. (2018) Mol. Ther. 26:289-303).

For sequential DNA FISH, slides were treated in 4% PFA, 0.5% TWEEN® 20 (polysorbate 20), and 0.5% NONIDET® P-40 (octylphenoxypolyethoxyethanol) for 10 minutes at room temperature, and then incubated in 0.2N HCl and 0.5% Triton™ X-100 for 10 minutes on ice. Following fixation, slides were denatured in 70% formamide 2X saline-sodium citrate at 80° C. for 10 minutes. Slides were then dehydrated in a graded alcohol series for 2 minutes each as for RNA FISH. Denatured probe (same labeled DNA as was used for RNA detection) was then applied to the slides and hybridized overnight at 37° C. (Namekawa, et al. (2010) Mol. Cell. Biol. 30:3187-3205). Washing and mounting of slides was the same as for RNA FISH. DNA FISH images were collected and analyzed by standard methods (Paulk, et al. (2018) Mol. Ther. 26:289-303).

### Example 2: AAV Vectors Contain Abundant Incorporation of Contaminant DNA Near REP Binding Elements

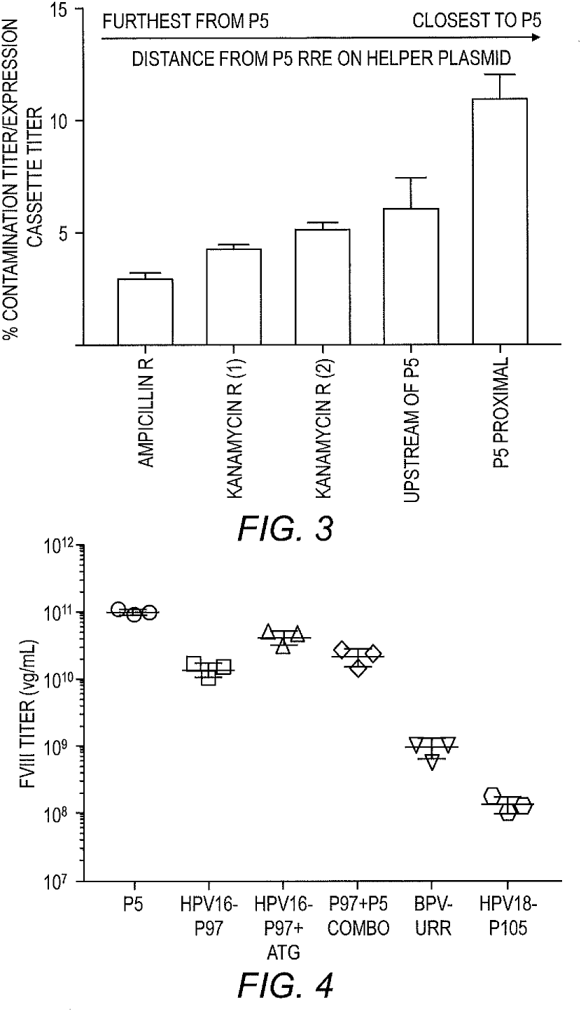

DNA contamination in AAV preparations has been identified in previous studies (Penaud-Budloo, et al. (2017) Hum. Gene Ther. Meth. 28 (3) : 148-162; Hauck, et al. (2009) Mol. Ther. 17:144-152; Chadeuf, et al. (2005) Mol. Ther. 12:744-753; Schnodt, et al. (2016) Mol. Ther. Nucl. Acids DOI: 10.1038/mtna. 2016.60) . Therefore, the nature of these contaminants was assessed in greater detail. Specifically, purified AAV was assessed by PCR analysis for amplicons in the plasmid backbone of the expression cassette plasmid and the presence of DNA outside of the expression cassette was observed. It was posited that the distribution of incorporated sequences from producer plasmids would not be uniform across the producer plasmids. Therefore, AAV DNA at increasing distances from the REP binding sites was examined. qPCR analysis showed that contaminant sequences closer to the inverted terminal repeats (ITRs) were more abundant than more remote sequences (FIG. 2). This indicated that contaminant incorporation was driven by an active process rather than passive uptake of plasmid DNA sequences by AAV virions. A possible mechanism for active incorporation of contaminant DNA would be through aberrant replication initiation at REP binding sites in the production plasmids. There is a REP binding site present in the P5 promoter, which controls expression of the large REP proteins (REP78 and REP68). Sequences upstream of the P5 promoter in the REP-CAP plasmids were examined to assess whether they had the potential to be actively incorporated into AAV virions. qPCR analysis of amplicons upstream of P5 showed abundant incorporation of contaminant DNA (FIG. 3). Sequences closer to the P5 REP binding site were present at greater levels than more distal sequences. To verify this phenotype, next generation sequencing was performed on AAV preparations and the reads were mapped back to the AAV production plasmids. The same pattern of DNA contamination was observed, with sequences adjacent to the ITRs and upstream of the P5 promoter being present in the highest abundance. Moreover, when analyzing a REPCAP plasmid, wherein the P5 promoter was moved to a position downstream of the CAP gene, the contamination phenotype followed the location of the P5 promoter. Together, these data indicated that AAV DNA contaminants from the producer plasmids was an active process correlated with the distance from REP binding elements. To ensure contamination levels were not influenced by AAV production harvest timepoint and conditions, AAV collected from the supernatant or cell lysates at 2, 3, or 7 days post-harvest was analyzed by qPCR, with no difference observed.

### Example 3: Contaminants Found in AAV Are Persistently Transferred and Transcribed Post-Infection

AAV genomes are known to persist in transduced cells in an episomal state (Penaud-Budloo, et al. (2008) J. Virol. 82:7875-7885). To determine if contaminant DNA sequences were present and active post-infection, an RNA/DNA FISH strategy was developed that would identify AAV contaminant sequences in cell nuclei. In infected Huh7 cells, both expression cassette and contaminant DNA were detectable at 48 hours post-infection. To examine if AAV could be transferred persistently in vivo, male C57BL/6 mice were injected with a high dose (8.4e11vg) of AAV8_scLP1hFIX or AAV8_ssHLP-hFVIII. Mouse livers were harvested at a short timepoint (1 week) or a long timepoint (4 months) post-infection and DNA recovered from mouse livers was assessed for the presence of contaminant sequences. Contaminant amplicons were detectable by qPCR. Contaminant DNA was also detected in the nucleus of mouse hepatocytes by DNA FISH at both short and long timepoints post-infection. Next generation sequencing showed that contaminants were efficiently transferred post-infection and present in both the short and long timepoints post-infection making up approximately 9% of virally mapped reads for FVIII and 1% of the reads for FIX, closely matching the profile of the viral preparation. To assess the potential of AAV contaminant sequences to be transcribed, infected Huh7 cells were analyzed. RNA FISH analysis of cells identified both vector DNA and contaminant transcripts. RNA FISH of expression cassette and transgene reads confirmed transcribed contaminant RNA in both 1-week and 4-month post-infection mouse hepatocytes. RT-PCR on AAV-infected mouse livers also confirmed the presence of transcript in mouse hepatocytes post-AAV infection. In mice at 4 months post-infection, RNAseq analysis identified contaminant RNA reads in a pattern that matched the DNA contamination phenotype. The reads made up approximately 3% of the total RNA detected from AAV origin in the FVIII-infected hepatocytes and 0.58% of the reads from the FIX-infected hepatocytes. FVIII infected mice were also DNA and RNA-seq analyzed at 1-week post-infection and showed transfer and transcriptional activity of contaminant sequences.

### Example 4: Contaminants in AAV Can Be Translated After AAV Infection

Previous RNA-seq studies of the wild-type AAV virus have revealed anti-P5 promoter activity, driving small RNAs (Stutika, et al. (2016) J. Virol. 90(3):1278-89; Stutika, et al. (2016) PLoS One 11:e0161454). It is possible that anti-P5 activity would be sufficient to drive RNA and protein expression of AAV contaminants. To test the potential for AAV contaminants to be translated, a fluorescent reporter system inserted upstream of the incorporation region in the P5 promoter was developed. 293T cells were transfected with REPCAP plasmids that contained either an upstream GFP cassette (P5-GFP), a GFP cassette in reverse (P5-GFP reverse), or no cassette (P5-empty). 293T cells transfected with P5-GFP plasmid expressed GFP, whereas cells transfected with P5-GFP reverse and P5-empty did not. Constructs were used to package an hFVIII cassette. 293T cells were then transduced with a range of multiplicity of infection (MOI) of each vector. After 72 hours, GFP-positive cells were observed in AAV8 P5-GFP transduced cells but not in those transduced with AAV8 P5-GFP reverse or AAV8 P5-empty virions. Infected cells were subjected to flow cytometry, which indicated that approximately 4% of the AAV8 P5-GFP cells were GFP positive.

Previous research has shown that in impure AAV preparations, protein contaminants from AAV production can be transferred along with the viral particle and ‘pseudotransduce’ the cell (Alexander, et al. (1997) Hum. Gene Ther. 8:1911-1920). To ensure that the observed effect was not an artifact of pseudotransduction, a time-course experiment with P5-GFP was conducted. The percent of GFP+ve cells increased from 48 hours to 72 hours post-infection, indicating that the present GFP protein was being nascently formed, and not a result of protein carryover. To test this contamination translation phenotype in a biologically relevant model, C57BL/6 mice were intravenously infected with 4.11elvg of the AAV8 P5 vectors and livers were harvested 1-week post-infection. Immunohistochemical staining for GFP protein revealed substantial numbers of positive hepatocytes in the AAV8 P5-GFP-treated mice, comparable with a lower dose AAV8ssCMV_GFP control (2e10vg). By comparison, GFP-positive hepatocytes were not present in mice infected with the AAV8 P5-GFP reverse vector or the AAV8 P5-empty vector.

### Example 5: AAV Contaminants Can Be Translated Independently of ITR Flanked Genomes Suggesting Integration Into the Host Genome

To ascertain whether the P5-based AAV contaminants acted independently of the expression cassette, the P5-GFP REPCAP was used to produce AAV8 in which no transgene plasmid was co-transfected during production (P5-GFP/TG-). qPCR titration of the purified virus showed the abundant presence of GFP DNA. 293T cells were then infected with equivalent contaminant doses of AAV8 P5-GFP/TG-, P5-GFP, P5-GFP reverse, or P5-empty, and measured by flow cytometry for GFP expression at various timepoints. This analysis indicated that P5-GFP reverse and P5-empty showed no GFP+ve cells at any timepoint post-infection. Interestingly, the P5-GFP/TG- showed an equivalent expression profile to P5-GFP, demonstrating that these contaminants do not require interaction with an AAV cassette for translational activity. At 3-weeks post-infection, GFP expression levels had stabilized in the P5-GFP/TG- and P5-GFP cells, suggestive of integration events. To investigate whether the infection kinetics matched that of standard AAV transduction, 293T cells were infected with AAV8 at a range of MOIs by either P5-GFP/TG- or an ITR-flanked, single-stranded GFP cassette drive by the cytomegalovirus promoter (ssCMV_GFP). At any given MOI, the number of GFP-positive cells was far greater in the ssCMV_GFP-infected samples as compared with P5-GFP/TG- samples, regardless of timepoint. Interestingly, the proportion of GFP-positive cells that stably produced GFP, compared to the number of GFP-positive cells at 72 hours, was far greater in the P5-GFP/TG- group (1 in 12.9±11.365) than the ssCMV_GFP group (1 in 192±27.6) indicating that a greater proportion of protein producing P5 contaminants are integrated into the cell as compared with standard AAV genome infection. However, when gDNA of 293T cells was analyzed for GFP copy number, both P5-GFP/TG- and ssCMV_GFP infected cells had similar levels of stably present GFP copies relative to the detected copies at 72 hours post-infection (P5-GFP/TG-, 1.20%±0.28; ssCMV_GFP, 0.88%+0.22), indicating that these non-ITR containing contaminants from P5 are integrating into the 293T cell genome at an equivalent rate to the ITR-flanked expression cassette.

### Example 6: Papilloma Early Promoter P5 Replacements Remove Contamination Phenotype

The presence of a REP binding site within the P5 promoter led to the suggestion that incorporation of contaminant DNA into AAV from the REP/CAP plasmid was an active process driven by REP binding. AAV production and replication is a finely balanced process. The P5 promoter drives expression of the large REP proteins REP78 and REP68 and relies on a negative feedback loop to inhibit its own activity during the AAV replication process (Kyostio, et al. (1994) J. Virol. 68:2947-2957; Pereira, et al. (1997) J. Virol. 71:1079-1088). The timing of P5 self-repression, in concert with the later expression of the shorter forms of REP from the P19 promoter, is essential to yield efficient AAV production. As such, previous attempts to swap out this promoter for constitutively active promoters have failed to produce high titer virus (Ogasawara, et al. (1988) Microbiol. Immunol. 42:177-185). It was posited that an alternative system for high-titered AAV production that did not incorporate DNA upstream of the P5 promoter could be developed if a negative feedback loop for P5 promoter activity was retained, and the REP78 DNA binding region was altered. The papilloma virus early promoter sequences were chosen as the basis for the P5 replacement promoter design. It is known that REP78 of AAV2 can inhibit the activity of early promoters of Papilloma viruses. In addition, papilloma virus early promoter sequences do not contain the GCTC DNA binding motif present in the REP binding sites of AAV or human genome AAV integration sites (Horer, et al. (1995) J. Virol. 69(9):5485-96; Zhan, et al. (1999) J. Biol. Chem. 274(44):31619-24). It was therefore hypothesized that the P5 promoter could be replaced by promoter designs that incorporated REP inhibitory elements from Papilloma virus variants, yielding high titers of AAV without resulting in active incorporation of upstream DNA. A series of AAV2_8 REPCAP constructs were prepared in which the P5 promoter was replaced by promoters that incorporated elements from either the HPV16-P97 promoter, the HPV18-P105 promoter, or the BPV upstream regulatory region (URR). P5 replacement REP-CAP plasmids were used to produce a single-stranded AAV8-HLP-hFVIII construct. qPCR titer analysis showed that all P5 replacement vectors tested, except for the HPV18-P105 construct, could produce AAV and that three designs produced AAV to the same log scale as the P5 driven AAV2_8 REPCAP (FIG. 4). P5 replacement AAV was assayed by qPCR for amplicons upstream of the REP78 promoter sequences. While the original P5-driven REPCAP showed abundant contamination, the replacement promoter reduced contamination upstream of the P5 configuration. Of note, replacement REPCAP constructs were able to infect cultured 293T cells. To ensure that this contamination phenotype was not serotype specific, the same approach was applied to AAV2 production. AAV2 production in both the P5 and P5 replacement system was again produced to the same log scale. However, qPCR analysis of the P5 replacement promoter system indicated a reduction in contaminant amplicon abundance to 10.3% ± 2.1. of the P5 production levels.

While the P5 replacement vectors produced virus to an equivalent log scale, the relative titer of these vectors did not exceed 50% of vectors harboring the original P5 promoter. To determine if this would influence contaminant proportions from other sources, amplicons outside of the ITRs on the expression cassette plasmid were analyzed. The raw amount of contamination from this source remained similar to P5 based production. However, the P5 replacement setup had a higher proportion of this contamination due to the reduced production efficiency. Furthermore, while next generation sequencing analysis of AAV8_hFVIII produced with the P5+P97combo promoter revealed no detectable initiation of active DNA contamination upstream of the P5 promoter, when compared for total contamination against an identical vector produced with the P5 promoter, it appeared that DNA contamination levels in this preparation were equivalent, due to an increase of contamination from outside of each ITR.

### Example 7: P5 REP Binding Site Is Required, but Not Sufficient to Cause P5-Based Contaminant Incorporation Into AAV

To show that the contaminant incorporation effect was directly related to the REP binding element in the P5 promoter, a promoter was created wherein the REP binding element of P5 was directly substituted for the REP binding site of the HPV16 P97 promoter (ATGTATAAAACTAAGGGCGT AACCGAAATCGGTTGAACCGAAA; SEQ ID NO:11). When virus was produced using this hybrid promoter, the titers produced were also within the same log range as the P5 driven AAV2_8 REPCAP. Furthermore, contamination observed upstream of the hybrid promoter was again significantly reduced. Subsequently, the reverse experiment was performed wherein the P5 REP binding sequence (GCCCGAGTGAGCACGC; SEQ ID NO: 12) was inserted into the P97 promoter, in place of the P97 REP binding site. This promoter also produced AAV to the same log as the original P5 promoter. Surprisingly, the contamination phenotype upstream of the promoter did not return, demonstrating that the presence of the P5 REP binding site alone is not sufficient to generate incorporation of DNA contaminant sequences into AAV.

### Example 8: P5 Spacer Vectors Produce Equivalent Titers of AAV

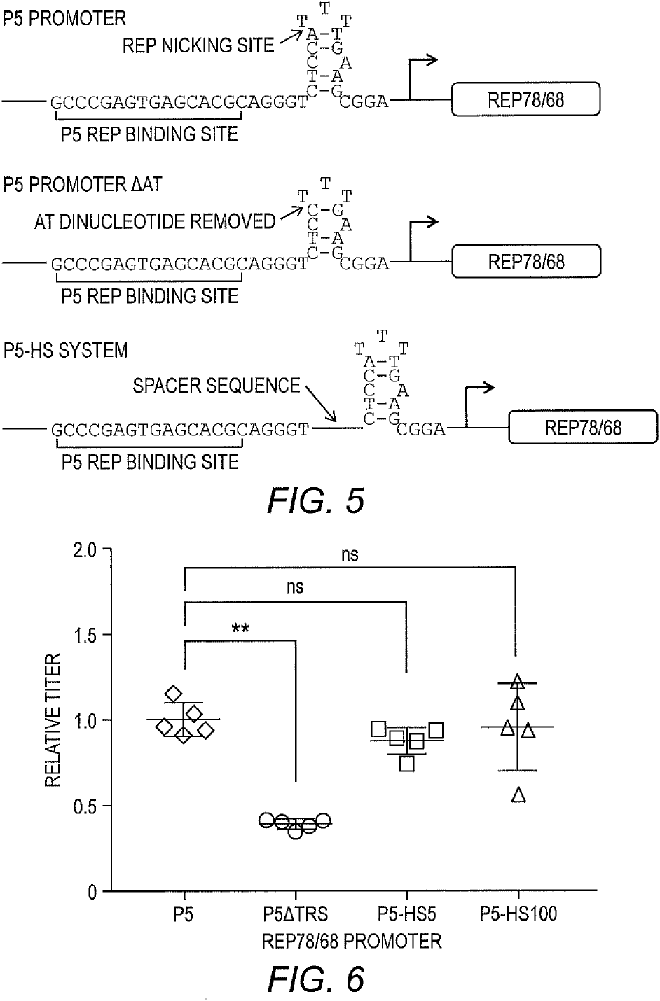

The P5 REP binding site is in proximity to a terminal resolution site (TRS) mimic dinucleotide sequence that exists within a YY1 box. It has been proposed that this YY1 box and adjacent sequence forms a loop from which the TRS mimic dinucleotide is positioned at the top of, allowing for replication from this site (Francois, et al. (2005) J. Virol. 79(17):11082-94). Previous work has shown that mutation of the P5 promoter at either the REP binding site, TATA box or either YY1 box results in significant reduction of promoter activity (Murphy, et al. (2007) J. Virol. 81:3721-30). Therefore, a series of P5 promoters were developed that retained all of these elements but yielded a physical separation between the YY1 box and the P5 REP binding site through the use of spacer sequences. These P5 “helper spacer” vectors (P5-HS) were compared to the P5 promoter with a mutation in the TRS mimic of the YY1 box (P5ΔTRS) (FIG. 5). The P5-HS vector significantly reduced DNA contamination directly upstream of P5, with a spacer of 5 bp reducing contaminant amplicon abundance to 15% ± 1 of the P5 system (P<0.001). The P5ΔTRS promoter also resulted in significant reduction of contamination (4.3% ± 0.58; P=0.0002), confirming that an intact YY1 box is concomitantly required with the P5 REP binding site to yield contamination, but again resulted in a lower viral production compared to P5 (38.6% ±3.1; P = 0.0015). REP gene contamination was not significantly different from P5 in either P5ΔTRS or the P5-HS system. Unlike the prior tested modifications, the P5-HS vectors yielded viral titers equivalent to that of the standard P5 configuration (P > 0.9999; FIG. 6). This was observed in designs of varying spacer length. A regression analysis of all vector constructs used to produce AAV8 FVIII showed that the relative contaminant levels from the expression cassette plasmid were indeed inversely correlated with the production efficiency, (R² = 0.749; P < 0.0001).

### Example 9: Large Scale AAV Production

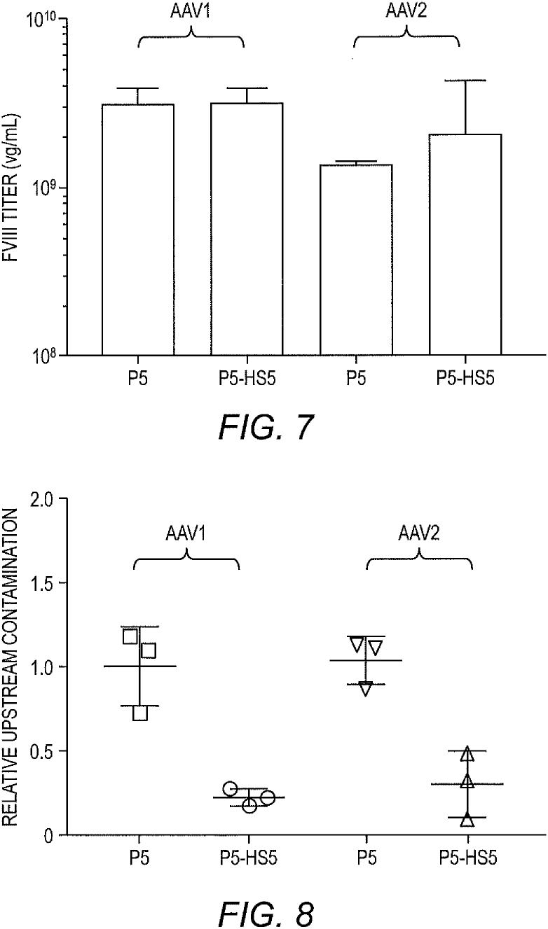

To test whether the P5-HS system could be applied to large scale AAV production, the 5 bp P5-spacer (P5-HS5) was tested at cell factory scale. Preparations of AAV8ssHLP_hFVIII and AAV8scLP1hFIXco were produced side-by-side using a plasmid in which REP78/68 was driven by either P5 or P5-HS5. AAV was purified and analyzed for contaminant abundance. Contaminant amplicons upstream of P5-HS5 were lower than P5 in both preparations whilst titer was equivalent. NGS reads mapped to the producer plasmids showed a large reduction of contamination upstream of P5 and an overall increase in purity. To ensure that P5-HS did not impact resultant AAV functionality, mice were infected with 2e10vg of AAV8ssHLP_hFVIII and FIX preps from P5 or P5-HS5 produced virus. Circulating protein levels were measured and shown to be equivalent between both production methods. Finally, to ascertain if P5-HS could be used to produce vector of serotypes other than AAV8, P5-HS5 was cloned into two additional serotype REP-CAP plasmids (AAV1 and AAV2), used to produce a ssHLPhFVIII construct, and measured for titer and upstream contamination. As expected, P5-HS5 helped produce AAV1 and AAV2 to equivalent titers as P5 (FIG. 7) and the examined upstream amplicons were significantly reduced in the P5-HS produced viruses of both serotypes (FIG. 8).

What is claimed is:

1. A recombinant P5 promoter comprising a REP binding site and a transcription start site-localized Ying-Yang 1 (YY1) binding site, wherein said P5 promoter comprises an exogenous spacer sequence inserted between the REP binding site and YY1 binding site.

2. The recombinant P5 promoter of claim 1, wherein the spacer is 5 nucleotides to 100 nucleotides in length.

3. A adeno-associated virus (AAV) vector comprising the recombinant P5 promoter of claim 1.

4. The AAV vector of claim 3, wherein said AAV is selected from the group of AAV1, AAV2, AAV3, AAV4, AAV6, AAV7, AAV8, and AAV9.

5. A host cell comprising the recombinant P5 promoter of claim 1.

6. A host cell comprising the AAV vector of claim 3.

7. A method of reducing DNA contamination upstream of a P5 promoter comprising inserting into a P5 promoter comprising a REP binding site and a transcription start site-localized Ying-Yang 1 (YY1) binding site, an exogenous spacer sequence between the REP binding site and YY1 binding site thereby reducing DNA contamination upstream of the P5 promoter.

8. A recombinant adeno-associated virus (AAV) harboring an HPV P97 promoter comprising a REP binding site and a transcription start site-localized Ying-Yang 1 (YY1) binding site.


SEQUENCE LISTING (12 sequences)

SEQ ID NO: 1 - DNA, 39 bp, adeno-associated virus 8
tatattaagcccgagtgagcacgcagggtctccattttg

SEQ ID NO: 2 - DNA, 38 bp, adeno-associated virus 6
tatttaagcccgagtgagcacgcagggtctccattttg

SEQ ID NO: 3 - DNA, 39 bp, adeno-associated virus 4
tatataaccgcgagtgagccagcgaggagctccattttg

SEQ ID NO: 4 - DNA, 39 bp, adeno-associated virus 3
tatatattctcgagtgagcgaaccaggagctccattttg

SEQ ID NO: 5 - DNA, 38 bp, adeno-associated virus 2; identical to SEQ ID NO: 2

SEQ ID NO: 6 - DNA, 38 bp, adeno-associated virus 1
tatatatggccgagtgagcgagcaggatctccattttg

SEQ ID NO: 7 - DNA, 38 bp, adeno-associated virus 8
gcccgagtgagcacgcagggtctccattttgaagcgga

SEQ ID NO: 8 - DNA, 36 bp, Artificial sequence
  Synthetic oligonucleotide
gcccgagtgagcacgcagggtctcctttgaagcgga

SEQ ID NO: 9 - DNA, 21 bp, adeno-associated virus 8
gcccgagtgagcacgcagggt

SEQ ID NO: 10 - DNA, 17 bp, adeno-associated virus 8
ctccattttgaagcgga

SEQ ID NO: 11 - DNA, 43 bp, Human papillomavirus type 16
atgtataaaactaagggcgtaaccgaaatcggttgaaccgaaa

SEQ ID NO: 12 - DNA, 16 bp, adeno-associated virus 8
gcccgagtgagcacgc